(19) United States
(12) Patent Application Publication
(10) Pub. No.: US 2025/0352826 A1
(43) Pub. Date: Nov. 20, 2025

(54) Installation of proton therapy equipment in existing radiotherapy treatment vaults

(71) Applicant: P-CURE LTD., Shilat Industrial Zone (IL)

(72) Inventors: Michael Marash, Rishon Lezion (IL); Udi Oren, Mishmar David (IL)

(21) Appl. No.: 18/871,825

(22) PCT Filed: Jun. 4, 2023
(86) PCT No.: PCT/IL2023/050573

§ 371 Date: Dec. 5, 2024

Related U.S. Application Data
(60) Provisional application No. 63/365,930, filed on Jun. 6, 2022.

(51) Int. Cl. A61N 5/10

(52) U.S. Cl. CPC A61N 5/1078; A61N 5/1043; A61N 5/1069; A61N 2005/1087; A61N 2005/1094

(57) ABSTRACT

Proton therapy treatment system to be deployed in an existing radiotherapy treatment vault such that radiation exposure is limited and meets radiation safety requirements. Patient support platform disposed in vault is configured for supporting patient, such as in a seated position. Imager is configured for imaging patient on patient support platform. A proton beam generator comprising a synchrotron is disposed in a region of vault. A proton beam delivery device is configured to deliver proton irradiation dose to isocenter of target tissue during treatment session, where delivery device may be gantry-less pencil beam scanning device operating without a collimator. Synchrotron may be disposed adjacent to entrance wall of vault and proton irradiation dose directed toward rear wall of vault. Synchrotron may extend through apertures of rear intermediate wall of vault and proton irradiation dose directed toward entrance intermediate wall of vault.

### FIELD OF THE INVENTION

The present invention generally relates to the fields of irradiation treatment, and to proton therapy equipment installation in particular.

### BACKGROUND OF THE INVENTION

Teletherapy is defined as a treatment methodology in which an irradiation source is at a distance from a body to be treated. X-rays and electron beams have long been used in teletherapy to treat various cancers. Unfortunately, X-rays exhibit a linear energy transfer approaching an exponential attenuation function and are therefore of minimal safe use for deeply embedded growths. The use of heavy particles, particularly hadrons and more particularly protons, in teletherapy, has found increasing acceptance, due to the ability of heavy particles to penetrate to a specific depth without appreciably harming intervening tissue. In particular, the linear energy transfer of hadrons exhibits an inversed depth profile with a marked Bragg peak defined as the point at which the hadrons deposit most of their energy and occurs at the end of the hadrons path. For electrons, the Bragg peak is not observable due to high scattering. For protons with energies below approximately 70 MeV, scattering considerably suppresses the Bragg peak. As a result of this effect, increased energy can be directed at an embedded growth as compared to X-rays and electron beams, which particularly harm intervening tissues. While the term hadrons include a wide range of particles, practically, protons and various ions are most widely used in therapy. For clarity, this document will describe treatment as being accomplished with protons, however this is not meant to be limiting in any way.

The protons or ions can be focused to a target volume of variable penetration depth. In this way the dose profile can be matched closely to the target volume with a high precision. In particular, a proton beam can conform to the shape and depth of a target growth, such as a tumor, so as to avoid irradiating healthy body tissue while delivering a lower total body irradiation dose. As a result, proton therapy can allow for escalated dosages as compared to conventional external beam therapies, which may be particularly beneficial for certain treatments, for example, ocular tumors or skull base and paraspinal tumors. Proton therapy may also enable high precision treatment plans with reduced side effects, such as for pediatric treatments or prostate cancer treatments. In order to ensure complete irradiation of a target growth, a plurality of beams arriving at the embedded growth from several different directions is usually applied. The point at which the plurality of beams intersects, whether they are beamed sequentially or simultaneously, is termed the “isocenter”. To maximize biological effectiveness, the isocenter must be precisely collocated with the target growth.

Irradiation treatment is performed on a target tissue in a well-defined process. In a first stage, known as a treatment planning stage, the target tissue is imaged and a treatment plan comprising dosage, patient position and irradiation angles are defined. Furthermore, placement markers may be defined, so as to ensure that subsequent irradiation sessions are properly targeted. Irradiation is then performed, responsive to the developed treatment plan, at a plurality of treatment sessions over a period of time, each session being known as a fraction. At each such fraction, care must be taken to ensure proper patient positioning, responsive to the placement markers, so as to avoid damage to organs in vicinity of the target tissue. Positioning of the patient responsive to the markers is performed based on visualization of the patient, responsive to the defined markers.

Particularly, during each fraction, the patient is positioned on a patient support platform, such as a bed, in a setup position. The setup position is identical to the patient position during the imaging of the treatment planning stage, except that it is in the treatment room and the center of the growth mass is positioned at the isocenter of the irradiation source. The setup position of the patient is optionally verified by imaging and/or positioning devices. Stabilization mechanisms may be applied to ensure patient positioning is maintained relative to the isocenter during the treatment, such as masks or shields to affix the face and/or other body parts of the patient.

Irradiation treatments are typically administered while the patient is in a lying or recumbent position, where the patient body is aligned substantially horizontal to the ground and supported by an underlying platform surface. For example, a recumbent positioned patient may be in a supine posture, with their back resting against the underlying surface and their face positioned upwards, or in a prone posture, with their chest against the underlying surface and their face pointed downwards. However, certain treatments may be difficult to perform on a horizontally positioned patient, such as due to the location of the growth mass in the body, and may require or be facilitated by an upright or non-horizontal positioning. Accordingly, the patient may be situated on a reclining chair that may be repositioned and reoriented along multiple axes in three-dimensional space, e.g., along six degrees of freedom. An upright or seated positioning may also provide greater patient comfort relative to a recumbent positioning, such as for patients suffering from breathing complications. Furthermore, upright positioning may affect changes in the volume, location, and/or motion of body organs, such as the lungs and heart, compared to recumbent positioning, which could have beneficial impacts in certain clinical situations.

Despite the various benefits, proton therapy remains marginally utilized in clinical settings. A primary reason relates to the sizable physical dimensions of the requisite equipment, which is infeasible to integrate into existing radiation therapy treatment facilities. Specifically, the equipment for generating and delivering proton-based irradiation, such as a particle accelerator, is considerably large and difficult to incorporate into a conventional radiotherapy treatment vault, often referred to colloquially as a linear accelerator (LINAC) vault. Furthermore, safety considerations are a critical factor for permitting clinical treatments using proton irradiation. Stringent measures are required to prevent harmful irradiation exposure, which further inhibits the installation of proton therapy equipment within conventional radiotherapy treatment vaults. For example, a proton accelerator generally needs to be installed in a separate shielded bunker within the facility, particularly cyclotron accelerators which produce harmful neutrons during operation. Therefore, the construction of a second large new vault is generally required to accommodate the proton therapy equipment.

### SUMMARY OF THE INVENTION

In accordance with one aspect of the present invention, there is thus provided a proton irradiation treatment system to be deployed in an existing radiation treatment vault. The system comprises a patient support platform, at least one imager, a proton beam generator, and a proton beam delivery device. The patient support platform is disposed in a region of the vault and configured for supporting a patient during a treatment session. The imager is configured for imaging the patient on the patient support platform. The proton beam generator comprises a synchrotron, disposed in a region of the vault, and configured to generate a proton irradiation beam. The proton beam delivery device is coupled with the synchrotron and disposed in a region of the vault, and is configured to deliver at least one proton irradiation dose to an isocenter of a target tissue of the patient with respect to an imaging coordinate system, during a proton therapy treatment, such that radiation exposure of the proton therapy treatment is limited and meets radiation safety requirements. The system may further comprise a controller, where during a treatment planning stage, the controller is configured to receive treatment prescriptions for treating the patient, to receive at least one image of the patient on the patient support platform, and a position and orientation of the patient respective of the image, and to generate a proton therapy treatment plan responsive to the received image and respective position and orientation and received treatment prescriptions, and where during a treatment application stage, the controller is configured to direct the proton beam delivery device to emit a proton irradiation dose, while selectively repositioning and reorienting a target tissue of the patient with respect to an isocenter of the proton irradiation beam, in accordance with treatment planning fields of the treatment plan. The treatment plan may comprise a series of irradiation dose parameters for at least one treatment session, each of the irradiation dose parameters comprising, for each respective irradiation does: a dosage, a position and orientation of the patient relative to an isocenter, and an irradiation angle. The proton beam delivery device may comprise a gantry-less pencil beam scanning (PBS) device, configured to deliver the proton irradiation dose without a collimator. The patient support platform may be a seatable platform, and wherein the patient is in a seated position during the treatment session. The imager may be disposed outside the vault. The synchrotron may be disposed adjacent to an entrance wall of the vault, and the proton beam delivery device may be configured to direct the proton irradiation dose toward the patient support platform disposed adjacent to a rear wall of the vault. The synchrotron may be disposed within an enclosed partition defined by an entrance wall of the vault, an outer side wall of the vault, and an inner side wall of the vault. The synchrotron may be disposed adjacent to a rear wall of the vault, and the proton beam delivery device may be configured to direct the proton irradiation dose toward the patient support platform disposed adjacent to an entrance wall of the vault. The synchrotron may be configured to extend through a plurality of apertures of a first intermediate inner wall of the vault adjacent to the rear wall, and the patient support platform may be disposed at a rear end of a second intermediate inner wall of the vault adjacent to the entrance wall. The vault may comprise at least one intermediate wall or door, comprising a concrete material having a thickness of at least 1 meter and density of at least 2.35 g/cm³. The vault may comprise at least one additional shielding layer of: an iron shielding of approximately 20 cm thickness on an interior side of the intermediate wall; a borated polyethylene shielding of approximately 40 cm thickness on an exterior side of the intermediate wall; and a borated polyethylene shielding of approximately 40 cm thickness on an interior side of an entrance door of the vault. The system may further comprise a platform adjustment mechanism, configured to adjust the platform translationally and rotationally about three orthogonal axes, to correspondingly alter a position or orientation of the patient along six degrees of freedom. The distance between an imaging isocenter of the patient during the treatment planning stage, and an imaging isocenter of the patient during the treatment application stage, may be less than 2 meters. The distance between a distal end of the proton beam delivery device and a skin portion of the patient, during the treatment application stage, may be less than 1 meter.

In accordance with another aspect of the present invention, there is thus provided a method for proton irradiation treatment in an existing radiation treatment vault. The method comprises the steps of: installing a patient support platform in a region of the vault, the patient support platform configured for supporting a patient; providing at least one imager, configured for imaging the patient on the patient support platform; installing a proton beam generator comprising a synchrotron, in a region of the vault, the synchrotron configured to generate a proton irradiation beam; and installing a proton beam delivery device, in a region of the vault, the proton beam delivery device configured to deliver at least one proton irradiation dose to an isocenter of a target tissue of the patient with respect to an imaging coordinate system, during a proton therapy treatment, such that radiation exposure of the proton therapy treatment is limited and meets radiation safety requirements. The method may further comprise the steps of, during a treatment planning stage, receiving treatment prescriptions for treating the patient, receiving at least one image of the patient on the patient support platform, and a position and orientation of the patient respective of the image, and generating a proton therapy treatment plan responsive to the received image and respective position and orientation and received treatment prescriptions, and during a treatment application stage, directing the proton beam delivery device to emit a proton irradiation dose, while selectively repositioning and reorienting a target tissue of the patient with respect to an isocenter of the proton irradiation beam, in accordance with treatment planning fields of the treatment plan. The treatment plan may comprise a series of irradiation dose parameters for at least one treatment session, each of the irradiation dose parameters comprising, for each respective irradiation does: a dosage, a position and orientation of the patient relative to an isocenter, and an irradiation angle. The proton beam delivery device may comprise a gantry-less pencil beam scanning (PBS) device, configured to deliver the proton irradiation dose without a collimator. The patient support platform may be a seatable platform, and wherein the patient is in a seated position during the treatment session. The imager may be disposed outside the vault. The synchrotron may be disposed adjacent to an entrance wall of the vault, and the proton beam delivery device may be configured to direct the proton irradiation dose toward the patient support platform disposed adjacent to a rear wall of the vault. The synchrotron may be disposed within an enclosed partition defined by an entrance wall of the vault, an outer side wall of the vault, and an inner side wall of the vault. The synchrotron may be disposed adjacent to a rear wall of the vault, and the proton beam delivery device may be configured to direct the proton irradiation dose toward the patient support platform disposed adjacent to an entrance wall of the vault. The synchrotron may be configured to extend through a plurality of apertures of a first intermediate inner wall of the vault adjacent to the rear wall, and the patient support platform may be disposed at a rear end of a second intermediate inner wall of the vault adjacent to the entrance wall. The vault may comprise at least one intermediate wall or door, comprising a concrete material having a thickness of at least 1 meter and density of at least 2.35 g/cm³. The vault may comprise at least one additional shielding layer of: an iron shielding of approximately 20 cm thickness on an interior side of the intermediate wall; a borated polyethylene shielding of approximately 40 cm thickness on an exterior side of the intermediate wall; and a borated polyethylene shielding of approximately 40 cm thickness on an interior side of an entrance door of the vault. The system may further comprise a platform adjustment mechanism, configured to adjust the platform translationally and rotationally about three orthogonal axes, to correspondingly alter a position or orientation of the patient along six degrees of freedom. The distance between an imaging isocenter of the patient during the treatment planning stage, and an imaging isocenter of the patient during the treatment application stage, may be less than 2 meters. The distance between a distal end of the proton beam delivery device and a skin portion of the patient, during the treatment application stage, may be less than 1 meter.

### BRIEF DESCRIPTION OF THE DRAWINGS

The present invention will be understood and appreciated more fully from the following detailed description taken in conjunction with the drawings in which:

FIG. 1 is a schematic illustration of a proton irradiation treatment system, constructed and operative in accordance with an embodiment of the present invention;

FIG. 2 is an illustration of an exemplary synchrotron configuration, constructed and operative in accordance with an embodiment of the present invention;

FIG. 3 is an illustration of a floor plan layout of an exemplary LINAC vault for deploying a proton irradiation treatment system, operative in accordance with an embodiment of the present invention;

FIG. 4 is a floor plan layout illustration of a first exemplary configuration for installing a proton irradiation treatment system in an existing LINAC vault where the proton beam is directed toward a rear wall of the vault, constructed and operative in accordance with an embodiment of the present invention; and

FIG. 5 is a floor plan layout illustration of a second exemplary configuration for installing a proton irradiation treatment system in an existing LINAC vault where the proton beam is directed toward an entrance wall of the vault, constructed and operative in accordance with an embodiment of the present invention.

### DETAILED DESCRIPTION OF THE EMBODIMENTS

The present invention overcomes the disadvantages of the prior art by providing a proton therapy treatment system and method that can be accommodated into an existing radiation therapy (radiotherapy) treatment vault. The disclosed embodiments allows existing health care facilities to incorporate proton therapy treatments without requiring significant infrastructure modifications while maintaining safety requirements in terms of radiation shielding, and further enabling the treatment of patients in a seated position.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

It will be understood that when an element is referred to as being “on”, “attached” to, “operatively coupled” to, “operatively linked” to, “operatively engaged” with, “connected” to, “coupled” with, “contacting”, “added to”, etc., another element, it can be directly on, attached to, connected to, operatively coupled to, operatively engaged with, coupled with, added to, and/or contacting the other element or intervening elements can also be present. In contrast, when an element is referred to as being “directly contacting” another element or “directly added” to another element, there are no intervening elements and/or steps present.

Whenever the terms “about” or “approximately” is used, it is meant to refer to a measurable value such as an amount, a temporal duration, and the like, and is meant to encompass variations from the specified value, as such variations are appropriate to perform the disclosed methods.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Whenever terms “plurality” and “a plurality” are used it is meant to include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

Throughout, this disclosure mentions “disclosed embodiments”, “disclosed systems” and “disclosed methods”, which refer to examples of inventive ideas, concepts, and/or manifestations described herein. The fact that some disclosed embodiments are described as exhibiting a feature or characteristic does not mean that other disclosed embodiments necessarily share that feature or characteristic.

This disclosure employs open-ended permissive language, indicating for example, that some embodiments “may” employ, involve, or include specific features. The use of the term “may” and other open-ended terminology is intended to indicate that although not every embodiment may employ the specific disclosed feature, at least one embodiment employs the specific disclosed feature.

The term “operator” is used herein to refer to any individual person or group of persons using or operating a method or system according to a disclosed embodiment, such as a medical practitioner involved in performing a proton therapy procedure (e.g., a radiation oncologist, a radiation therapy nurse, a medical radiation physicist, a radiation therapist, a dosimetrist, and the like).

The terms “subject” and “patient” are used interchangeably herein to refer to an individual upon which a method or system according to a disclosed embodiment is performed, such as a person undergoing a proton therapy procedure. The subject may be any living entity, such as a person, human or animal, characterized with body tissue subject to irradiation treatment.

The terms “proton therapy” and “proton treatment” are used interchangeably herein to broadly encompass all forms of particle therapy or hadron therapy that applies beams of energized ionizing particles for radiotherapy purposes, including but not limited to protons, neutrons and other types of ions (all of which are considered encompassed herein by the term “protons”).

The present invention will be apparent from the following detailed description, which proceeds with reference to the accompanying drawings, wherein the same references relate to the same elements. For a better understanding of certain embodiments and to show how the same may be carried into effect, reference will now be made, purely by way of example, to the accompanying drawings in which like numerals designate corresponding elements or sections throughout.

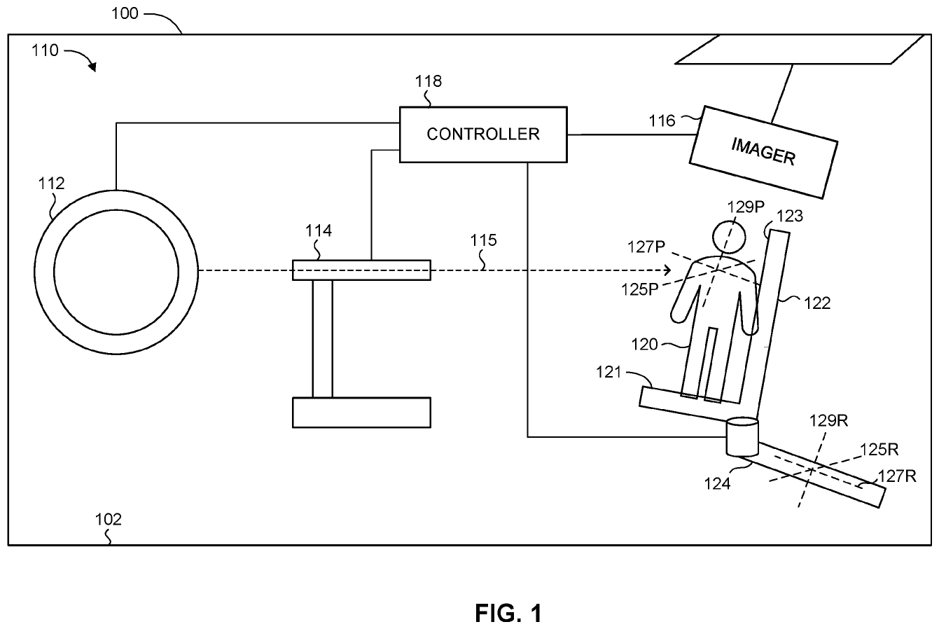

Reference is now made to FIG. 1, which is a schematic illustration of a proton irradiation treatment system, generally referenced 110, constructed and operative in accordance with an embodiment of the present invention. The components of system 110 are configured to be deployed in an existing conventional radiotherapy treatment vault 100, also referred to as a linear accelerator (LINAC) vault. Treatment system 110 includes a proton beam generator 112, a proton beam delivery device 114, at least one imager 116, a controller 118, a patient support platform 122, and a platform adjuster 124. Controller 118 is communicatively coupled with proton beam generator 112, with proton beam delivery device 114, with imager 116, and with platform adjuster 124. LINAC vault 100 is a shielded room containing at least proton beam generator 112, proton beam delivery device 114, and patient support platform 122.

Patient support platform 122 is configured for supporting a patient 120 during a treatment session or planning phase. In one embodiment, patient support platform 122 includes a chair, such that patient 120 may be in a sitting position and supported by a seat 121 and a back rest 123 (as illustrated in FIG. 1). Patient support platform 122 may also include or be converted into a bed, such that patient 120 may be in a lying or recumbent position (i.e., horizontal to the ground) supported by the bed.

Patient support platform 122 may be adjusted using a platform adjuster 124, so as to correspondingly alter a position and/or orientation of patient 120 along six degrees of freedom (6DOF). Platform adjuster 124 may include a rotational adjustment mechanism configured to adjust at least one rotational angle of platform 122 (e.g., pitch, yaw, roll rotations) and/or a translational adjustment mechanism configured to translationally displace platform 122 along at least one axis. For example, platform adjuster 124 may include a first mechanism for adjusting a height of platform 122, and a second mechanism for rotating platform 122 about pitch, yaw, and roll axes, respectively (e.g., causing patient 120 to lay back, tip sideways, or swivel, respectively). For example, a rotational adjustment mechanism may rotate platform 122 about three orthogonal axes 125R, 127R, 129R, where a first axis 125R is parallel to a floor 102 of the treatment vault room 100, a second axis 127R is parallel to floor 102 and orthogonal to first axis 125R, and a third axis 129R is orthogonal to floor 102. The rotation of patient support platform 122 causes a rotation of patient 120 about three orthogonal axes 125P, 127P, 129P, where a first axis 125P is orthogonal to a longitudinal axis of seat 121 and orthogonal to a longitudinal axis of back rest 123, a second axis 127P is parallel to a longitudinal axis of seat 121, and a third axis 129P is parallel to a longitudinal axis of back rest 123. In one embodiment, axes 125P, 127P and 129P correspond to axes 125R, 127R and 129R, respectively.

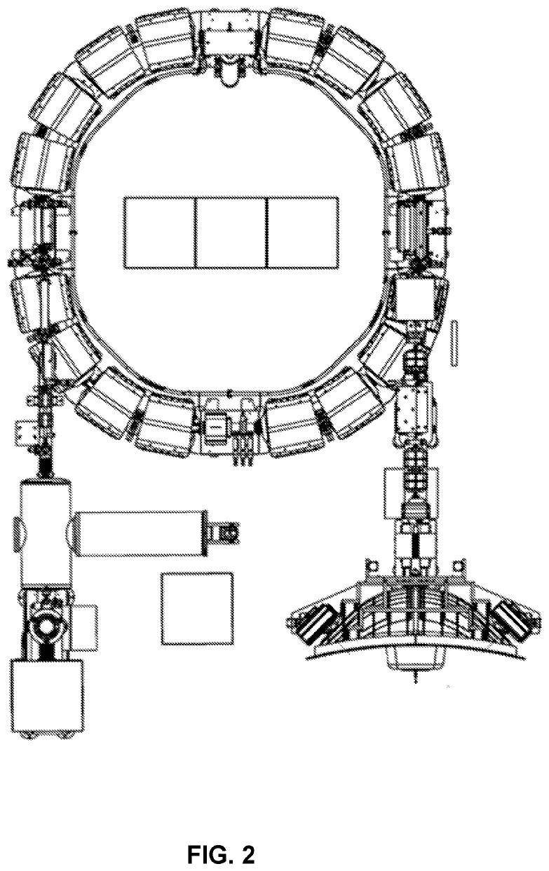

Proton beam generator 112 includes components and techniques for generating an irradiation therapy proton beam, such as a particle accelerator. According to one embodiment, proton beam generator 112 includes a synchrotron. A synchrotron is a type of circular particle accelerator in which charged particles (e.g., protons) are accelerated through a sequence of magnets around a fixed closed-loop path. The magnetic field bending the particle beam around the circular loop increases over time as it is synchronized to the kinetic energy of the particles (hence the term “synchrotron”). In contrast to a cyclotron type of particle accelerator, a synchrotron generally does not produce harmful neutron radiation during its operation and can thus be safely placed within treatment vault 100. While synchrotrons are generally larger in size than cyclotrons, newer synchrotron models are more compact, which may facilitate its transport and delivery (e.g., via regular elevators) and allow for its installation within an existing LINAC vault. Accordingly, the dimensions of the synchrotron may be selected to facilitate its deployment within vault 100. For example, the synchrotron may be characterized by an outer ring diameter of about 5 meters (m), and an accelerator weight of about 15 tons. Although a synchrotron may be limited to a pulsed beam operation mode, a synchrotron may be capable of delivering any desired energy level (up to a maximum threshold) and may provide efficient beam usage in comparison to a cyclotron. Exemplary operating parameters of the synchrotron may include: a proton energy range of about 30-330 MeV; a treatment energy range of about 70-250 mega electron volts (MeV); an extracted beam intensity of about 2.5×10⁹ protons per cycle; a magnetic field strength at injection of approximately 90 milliTesla (mT); a magnetic field strength of approximately 1.9 Tesla (T); and an average energy consumption during treatment of approximately 30 kilowatts (kW). Reference is made to FIG. 2, which is an illustration of an exemplary synchrotron configuration, constructed and operative in accordance with an embodiment of the present invention. A synchrotron may also be deployed within a conventional LINAC vault having an intermediate wall, by providing apertures in the wall to accommodate segments of the synchrotron loop, without requiring the complete removal of the wall, as will be discussed further hereinbelow with reference to FIG. 5.

Proton beam delivery device 114 includes components and techniques for delivering an irradiation dose 115 to patient 120 from the generated proton beam. According to one embodiment, proton beam delivery device 114 includes a gantry-less pencil beam scanning delivery device. Pencil beam scanning (PBS) involves steering the proton beam along a target area in a set pattern and focused beam shape that is typically only a few millimeters wide (i.e., the width of a pencil), using an electronically and/or magnetically guided scanning mechanism. PBS allows controlling the beam position and depth for more precisely delivering the radiation dose to the target tissue (e.g., tumor) in three dimensions. In particular, PBS may direct the proton beam in a customized pattern based on the location, shape and size of a tumor so as to precisely treat the tumor while avoiding nearby healthy tissue. Compared to other proton delivery techniques, such as passive scattering proton therapy (PSPT), PBS provides a smaller beam width and controlled scanning and allows for delivering intensity-modulated dosages to further minimize harmful exposure to healthy tissue and ensuring high quality treatment in accordance with prescribed treatment plan objectives and constraints. PBS also does not require use of specialized components, such as apertures or compensators, for the proton beam delivery, which can help reduce treatment time and delays, minimize costs, and increase flexibility. Furthermore, the PBS delivery device does not utilize collimators that may produce harmful neutron radiation.

The PBS device may be characterized with beam extraction, steering magnets (along x-axis and y-axis) and beam detection with a luminesce detector and ion chamber array. The treatment field size may be at least 30×30 cm (e.g., 40×40 cm). The PBS device may include a removable Faraday cap for routine calibration. The PBS device may provide intensity-modulated radiation therapy (IMRT) capability.

Proton beam delivery device 114 may also operate without requiring a rotating gantry for positioning and directing the beam, thereby significantly minimizing the required equipment and augmenting the feasibility of deploying system 110 in existing radiotherapy treatment vault 100. A gantry-less delivery mechanism allows for proton beam delivery to a wide variety of anatomical sites and body parts. Furthermore, the lack of a gantry allows for upright or seated positioning of the patient during both treatment and delivery. A seated position may reduce body motion allowing for more precise beam delivery and less organ motion, leading to more accurate treatment. In addition, the required setup time is shortened for patients in a seated position.

Imager 116 is configured for imaging patient 120, such as during and/or prior to a treatment session (i.e., during a treatment planning stage). For example, imager 116 may be a medical imaging device used in a medical treatment setting, including but not limited to: a computed tomography (CT) imager, a four-dimensional computed tomography (4DCT), an X-ray computed tomography (X-ray CT) scanner, an optical coherence tomography (OCT) scanner, a magnetic resonance imaging (MRI) scanner, and an ultrasound imager. In general, imager 116 may include any type of imaging sensor capable of acquiring and storing an image representation of an object or scene. Accordingly, the term “image” as used herein refers to any form of output from such an imager, including any optical or digital representation of a scene acquired at any wavelength or spectral region, and encompasses both a single image frame and a sequence of image frames (i.e., a “video image”). An image rotation mechanism (not shown) may be configured to rotate imager 116 about at least one axis, to enable imaging from selected directions or viewing angles. According to an embodiment, imager 116 is situated outside vault 100.

Controller 118 is configured to selectively control the operation of components of system 110 and may dynamically adjust operational parameters thereof. Controller 118 is further configured to receive instructions and data from components of system 110 and to perform required data processing.

Information may be conveyed between the components of system 110 over any suitable data communication channel or network, using any type of channel or network model and any data transmission protocol (e.g., wired, wireless, radio, WiFi, Bluetooth, and the like). The components and devices of system 110 may be based in hardware, software, or combinations thereof. It is appreciated that the functionality associated with each of the devices or components of system 110 may be distributed among multiple devices or components, which may reside at a single location or at multiple locations. For example, the functionality associated with controller 116 may be distributed between separate components, such as at least one control unit and at least one processing unit (e.g., which may be part of a server or a remote computer system accessible over a communications network, such as a cloud computing platform). Controller 116 may also be at least partially integrated with other components of system 110 (such as incorporated within a dedicated local control unit).

System 110 may optionally include and/or be associated with additional components not shown in FIG. 1, for enabling the implementation of the disclosed subject matter. For example, system 110 may include a user interface (not shown) for allowing a user to provide instructions or control various parameters or settings associated with the components of system 110, a display device (not shown) for visually displaying information relating to the operation of system 110, and/or memory or storage unit (not shown) for temporary storage of images or other data.

In operation, patient 120 is supported by platform 122. In one embodiment, patient 120 is in a seated position. During a first stage, referred to as a treatment planning stage, imager 116 images a target tissue of patient 120 to be treated, such as from a plurality of imaging angles. Controller 116 receives treatment prescriptions for a treatment of patient 120, such as details relating to the target tissue and recommended doses (e.g., recommended minimum and/or maximum doses) to be applied to the target tissue. Controller 116 determines a proton therapy treatment plan in accordance with the received treatment prescriptions and the received images. The treatment plan may include a series of irradiation dose parameters for at least one treatment session, defined at least by a dosage, a position and orientation of the patient (relative to an isocenter), and an irradiation angle for each irradiation dose. Controller 116 may construct a three-dimensional model of the target tissue based on the captured images to assist in determining the treatment plan. Placement markers may be positioned on or around patient support platform 122 to ensure proper targeting for each irradiation dose and to avoid harming organs or tissue in the vicinity of the target tissue. In a subsequent treatment stage, an operator applies proton irradiation to patient 120, during at least one treatment session, in accordance with the established treatment plan. Prior to the treatment, patient 120 is positioned in relation to the placement markers, such as based on a visualization of patient 120 responsive to the placement markers. In particular, patient 120 is placed in a setup position on platform 122 in the treatment room, the setup position corresponding to the patient position during the imaging performed in the treatment planning stage, such that the target tissue is centered at an isocenter of proton beam generator 112 and proton beam delivery device 114 when patient 120 is in the setup position. The setup position of patient 120 may be verified using auxiliary imaging and/or positioning devices. For example, a plurality of x-ray imaging devices may be integrated into the walls or floor of the treatment room and used for verifying proper patient positioning. Further imaging may also be carried out, such as using in-room CT modalities configured for imaging a patient 120 in a seated position. It is noted that the auxiliary imaging may further be utilized to ensure that the characteristics of the target tissue has not changed dramatically since the onset of treatment, in addition to verifying proper patient positioning. Stabilization mechanisms may be applied to ensure patient positioning is maintained relative to the isocenter during the treatment, such as a mask or shield to affix the face and/or other body parts of patient 120.

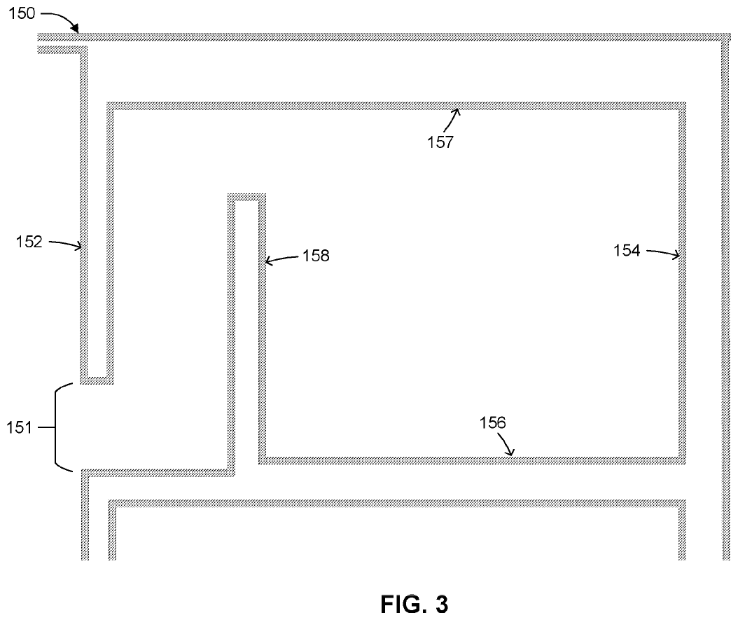

System 110 is configured to be installed in an existing radiotherapy treatment vault or LINAC vault. Reference is made to FIG. 3, which is an illustration of a floor plan layout of an exemplary LINAC vault, generally referenced 150, for deploying a proton irradiation treatment system, operative in accordance with an embodiment of the present invention. Vault 150 may be an existing structure (e.g., previously utilized for other purposes) or may be specifically constructed, such as within a radiation oncology facility, for accommodating system 110 to enable proton therapy treatment at the facility. Vault 150 is characterized by an entrance 151, an entrance wall 152, a rear wall 154, side walls 156, 157, and an intermediate wall 158. When system 110 is deployed in vault 150, the distance between the imaging isocenter and the treatment isocenter may be less than 2 meters, and the distance between the distal end of the nozzle of proton beam delivery device 114 and the skin of patient 120 (at which the irradiation dose is directed) may be less than 1 meter. At least some of the components of system 110 may be situated outside vault 150, such as imager 116.

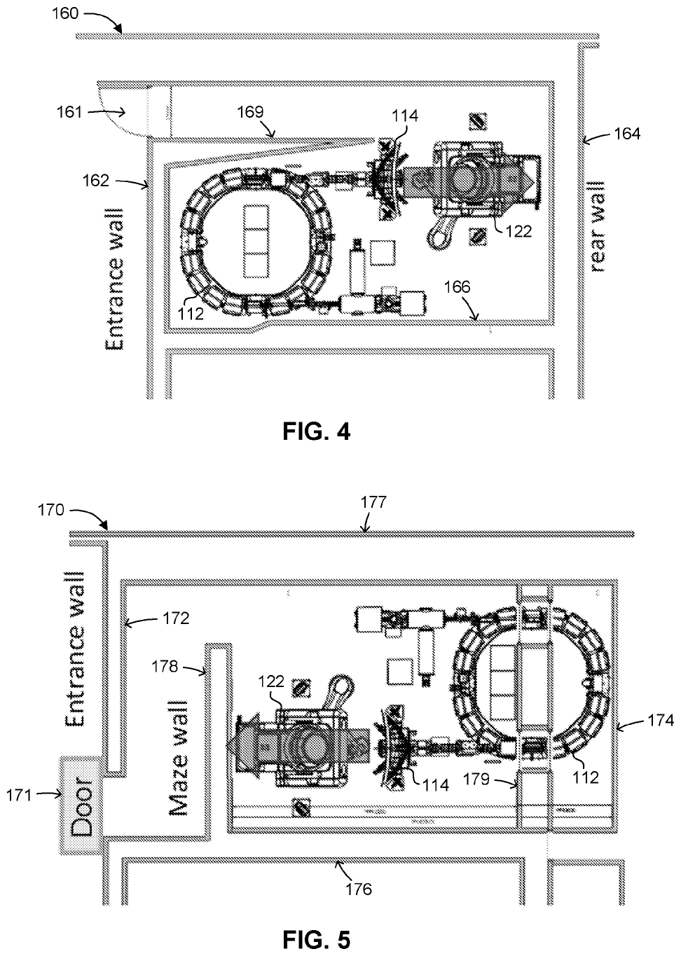

The components of system 110 may be deployed within vault 100 in various configurations. For example, proton beam generator 112 and delivery device 114 may be positioned at an entrance of the vault (i.e., the end where the room entrance is located), and configured to direct proton irradiation to a patient 120 positioned at a rear of the vault (i.e., the opposite end from where the room entrance is located). Reference is made to FIG. 4, which is a floor plan layout illustration of a first exemplary configuration for installing a proton irradiation treatment system in an existing LINAC vault, referenced 160, where the proton beam is directed toward a rear wall of the vault, constructed and operative in accordance with an embodiment of the present invention. Vault 160 is characterized by an entrance 161, an entrance wall 162, a rear wall 164, an outer side wall 166, and an inner side wall 169. Entrance wall 162 and rear wall 164 extend along a transverse axis of vault room 160, and side walls 166, 169 extend along a longitudinal axis of vault room 160, such that outer side wall 166, entrance wall 162, and inner side wall 169 define an enclosed cubicle or partitioned area adjacent to the entrance of vault 160. A synchrotron proton beam generator 112 is positioned within the defined partitioned area adjacent to the entrance wall of vault room 160 Patient support platform 122 is positioned at rear wall 164 of vault 160, i.e., at the far end opposite from entrance wall 162. Proton beam delivery device 114 is positioned at a middle location of vault 160 between synchrotron 112 and platform 122. The irradiation treatment is directed to patient 120 positioned on platform 122 at rear wall 164. Accordingly, vault 160 is able to fully accommodate system 110, while the partitioned cubicle area (defined by entrance wall 162 and side walls 166, 169) serves as a shielding, preventing radiation from penetrating beyond the treatment area and outside of vault 160. Furthermore, the irradiation treatment is directed toward the rear of vault 160 and away from the direction of potential bystanders, such as clinic staff members, who may be situated at or near the entrance.

In an alternative arrangement, proton beam generator 112 and delivery device 114 may be positioned at the rear of the vault and configured to direct proton irradiation to a patient 120 positioned at the entrance of the vault. Reference is made to FIG. 5, which is a floor plan layout illustration of a second exemplary configuration for installing a proton irradiation treatment system in an existing LINAC vault, referenced 170, where the proton beam is directed toward an entrance wall of the vault, constructed and operative in accordance with an embodiment of the present invention. Vault 170 is characterized by a door 171, an entrance wall 172, a rear wall 174, outer side walls 176, 177, and intermediate inner walls 178, 179. Outer side walls 176, 177 extend along a longitudinal axis of vault room 170, and intermediate inner walls 178, 179 extend along a transverse axis of vault room 170. First intermediate inner wall 178 (depicted in FIG. 5 as a “maze wall”) is arranged near the vault entrance adjacent to entrance wall 172, and second intermediate inner wall 179 is arranged at the rear adjacent to rear wall 174. A synchrotron proton beam generator 112 is positioned around second intermediate wall 179 adjacent to rear wall 174, at an opposite end from door 171. In particular, the synchrotron 112 is configured such that a first synchrotron segment (i.e., defining a segment of a closed loop) extends through a first aperture of intermediate wall 179, and a second synchrotron segment (i.e., defining another segment of the closed loop) extends through a second aperture of intermediate wall 179. In this manner, synchrotron 112 can be accommodated in vault room 170 having at least one transversely aligned intermediate inner wall without requiring the complete removal or dismantling of the intermediate inner wall, but rather by simply creating suitably sized apertures in the intermediate wall (i.e., while leaving the remainder of the intermediate wall intact). Patient support platform 122 is positioned at a rear side of first intermediate wall 178 (i.e., the side away from entrance wall 172 and door 171). Proton beam delivery device 114 is positioned at a middle location of vault 170 between synchrotron 112 and platform 122. The irradiation treatment is directed to patient 120 positioned on platform 122 at intermediate wall 178. Accordingly, vault 170 is able to fully accommodate system 110 without substantial changing the inner vault structure, while the intermediate wall 178 serves as a shielding, preventing radiation from penetrating beyond the treatment area and outside of vault 170. In effect, radiation would need to pass through two intermediate inner walls 178, 179 of vault room 170, further limiting radiation penetration and potential exposure.

The disclosed proton irradiation treatment system 110 may be deployed in an existing radiation treatment vault 100 while minimizing harmful secondary radiation exposure relating to neutron radiation, in accordance with safety regulations. Neutrons dominate in the prompt radiation field produced by protons. A neutron spectrum has two maxima: a first maxima around 100 MeV (due to the interaction of high-energy incident protons with a single nucleon), and a second maxima around 1-2 MeV (due to an evaporation of nucleons after the energy of the incident nucleon is distributed among other nucleons). The shape of the spectrum is rather independent of the location within the shield, the incident energy, or the shielding material (for the same hydrogen content). The primary processes for fast neutrons include elastic scattering (n,n) for lower energies, and inelastic processes, such as inelastic scattering (n, n′) and (n, 2n) reactions. The cross sections of inelastic processes increase with increasing mass number. Inelastic processes reduce neutron energy. Fast neutron shielding using high-Z (high-A) material (e.g., iron, lead) must be followed by hydrogen-containing shielding for low-energy neutrons. Accordingly, the radiation exposure of the proton therapy treatment of the disclosed embodiments is limited and meets radiation safety requirements when deployed in a LINAC vault. Such safety requirements may be based on local regulations, such as, for example, a maximum allowable (accrued) radiation dose of 2 milliSievert per year (msV/y) for the general public, and 20 msV/y for occupational exposure for those designated as “radiation workers”. Radiation safety requirements may alternatively or additionally be based on international regulations, such as, for example, a maximum allowable (accrued) radiation dose of 5 msV/y for the general public, and 50 msV/y for those designated as radiation workers.

Monte Carlo simulations were carried out to compare different options of proton therapy system configurations for accommodation into an existing radiation treatment (LINAC) vault, and to provide necessary shielding parameters. The simulation setup included a scanning proton beam and a CT stand placed on the beamline. A 40×40×40 water phantom was positioned at the isocenter. A 40×40 cm² field size at isocenter was conducted using a pencil beam with a 0.3 cm spot size (one sigma). During the simulations, 4×10⁷ initial proton histories were played. An effective dose distribution was calculated using flux-does conversion coefficients. The workload corresponded to 30 daily treatments with 2 Gy/Liter dose from protons.

A first group of Monte Carlo simulations for proton dose assessment was carried out for a 70 MeV proton beam and a 240 MeV proton beam. It is apparent that the 250 MeV operation mode has a substantially higher dose load. The dose at the entrance is formed primarily by backscattered neutrons. It can be conjectured that no extra shielding at the entrance is necessary. However, it may be necessary to ensure that a door is provided at the vault entrance. The area with a higher dose load is located beside the rear wall. An annual dose level outside the door and entrance wall is less than 2 msV for the 250 MeV operation mode.

A second group of Monte Carlo simulations for proton dose assessment was carried out for a 70 MeV proton beam and a 240 MeV proton beam. These simulations were applied with an intermediate inner wall of 1 m concrete (2.35 g/cm³). It appears that additional shielding may be required. Such additional shielding may include: a 20 cm iron shielding on an inner side of intermediate wall 168, a 40 cm borated polyethylene shielding on an outer side of intermediate wall 168, and/or a 40 cm borated polyethylene layer inside the entrance door of vault room 165. The annual dose level outside the door and entrance wall of room 165 is less than 2 msV for the 250 MeV operation mode.

A third group of Monte Carlo simulations for proton dose assessment was carried out for a 70 MeV proton beam and a 240 MeV proton beam. It is apparent that neutrons are the primary contribution to the secondary radiation field. Accommodation of the disclosed system 110 may require further protection against such neutron radiation.

While certain embodiments of the disclosed subject matter have been described, so as to enable one of skill in the art to practice the present invention, the preceding description is intended to be exemplary only. It should not be used to limit the scope of the disclosed subject matter, which should be determined by reference to the following claims.

1. A proton irradiation treatment system, configured to be deployed in an existing radiotherapy treatment vault, the system comprising:
a patient support platform, disposed in a region of the vault away from a vault entrance, for limiting harmful neutron radiation leaving the vault, the patient support platform being a seatable platform configured for supporting a patient in an upright or seated positioning during a treatment session;
at least one imager, disposed in the vault between the patient support platform and the vault entrance such that the imager forms a barrier to limit harmful neutron radiation leaving the vault, the imager configured for imaging the patient in an upright or seated positioning on the patient support platform;
a proton beam generator, disposed in the vault, the proton beam generator comprising a synchrotron, configured to generate a proton irradiation beam having limited harmful neutron radiation; and
a proton beam delivery device, coupled with the synchrotron and disposed in the vault, the proton beam delivery device comprising a gantry-less pencil beam scanning (PBS) device, configured to deliver at least one proton irradiation dose without a collimator and having limited harmful neutron radiation to an isocenter of a target tissue of the patient with respect to an imaging coordinate system, during a proton therapy treatment.

2. The system of claim 1, further comprising a controller, wherein during a treatment planning stage, the controller is configured to receive treatment prescriptions for treating the patient, to receive at least one image of the patient on the patient support platform, and a position and orientation of the patient respective of the image, and to generate a proton therapy treatment plan responsive to the received image and respective position and orientation and received treatment prescriptions, the treatment plan comprising a series of irradiation dose parameters for at least one treatment session, each of the irradiation dose parameters comprising, for each respective irradiation dose: a dosage, a position and orientation of the patient relative to an isocenter, and an irradiation angle, and
wherein during a treatment application stage, the controller is configured to direct the proton beam delivery device to emit a proton irradiation dose, while selectively repositioning and reorienting a target tissue of the patient with respect to an isocenter of the proton irradiation beam, in accordance with treatment planning fields of the treatment plan.

3. The system of claim 1, wherein the synchrotron is disposed adjacent to an entrance wall of the vault, and wherein the proton beam delivery device is configured to direct the proton irradiation dose toward the patient support platform disposed adjacent to a rear wall of the vault.

4. The system of claim 3, wherein the synchrotron is disposed within an enclosed partition defined by an entrance wall of the vault, an outer side wall of the vault, and an inner side wall of the vault.

5. The system of claim 1, wherein the synchrotron is disposed adjacent to a rear wall of the vault, and wherein the proton beam delivery device is configured to direct the proton irradiation dose toward the patient support platform disposed at a rear side of a first intermediate inner wall of the vault.

6. The system of claim 5, wherein the synchrotron is configured to extend through a plurality of apertures of a second intermediate inner wall of the vault adjacent to the rear wall.

7. The system of claim 1, wherein the vault comprises at least one intermediate wall or door, comprising a concrete material having a thickness of at least 1 meter and density of at least 2.35 g/cm³.

8. The system of claim 7, wherein the vault comprises at least one additional shielding layer selected from the group consisting of:
an iron shielding of approximately 20 cm thickness on an interior side of the intermediate wall;
a borated polyethylene shielding of approximately 40 cm thickness on an exterior side of the intermediate wall; and
a borated polyethylene shielding of approximately 40 cm thickness on an interior side of an entrance door of the vault.

9. The system of claim 1, further comprising a platform adjustment mechanism, configured to adjust the platform translationally and rotationally about three orthogonal axes, to correspondingly alter a position or orientation of the patient along six degrees of freedom.

10. The system of claim 2, wherein a distance between an imaging isocenter of the patient during the treatment planning stage, and an imaging isocenter of the patient during the treatment application stage, is less than 2 meters.

11. The system of claim 1, wherein a distance between a distal end of the proton beam delivery device and a skin portion of the patient, is less than 1 meter.

12. A method for deploying a proton irradiation treatment system in an existing radiotherapy treatment vault, the method comprising the steps of:
installing a patient support platform in a region of the vault away from a vault entrance, for limiting harmful neutron radiation leaving the vault, the patient support platform being a seatable platform configured for supporting a patient in an upright or seated positioning during a treatment session;
installing at least one imager in the vault between the patient support platform and the vault entrance such that the imager forms a barrier to limit harmful neutron radiation leaving the vault, the imager configured for imaging the patient in an upright or seated positioning on the patient support platform;
installing a proton beam generator in the vault, the proton beam generator comprising a synchrotron, configured to generate a proton irradiation beam having limited harmful neutron radiation; and
installing a proton beam delivery device, coupled with the synchrotron, in the vault, the proton beam delivery device comprising a gantry-less pencil beam scanning (PBS) device, configured to deliver at least one proton irradiation dose without a collimator and having limited harmful neutron radiation to an isocenter of a target tissue of the patient with respect to an imaging coordinate system, during a proton therapy treatment.

13. The method of claim 12, wherein the synchrotron is disposed adjacent to an entrance wall of the vault, and wherein the proton beam delivery device is configured to direct the proton irradiation dose toward the patient support platform disposed adjacent to a rear wall of the vault.

14. The method of claim 13, wherein the synchrotron is disposed within an enclosed partition defined by an entrance wall of the vault, an outer side wall of the vault, and an inner side wall of the vault.

15. The method of claim 12, wherein the synchrotron is disposed adjacent to a rear wall of the vault, and wherein the proton beam delivery device is configured to direct the proton irradiation dose toward the patient support platform disposed at a rear side of a first intermediate inner wall of the vault.

16. The method of claim 15, wherein the synchrotron is configured to extend through a plurality of apertures of a second intermediate inner wall of the vault adjacent to the rear wall.

17. The method of claim 12, wherein the vault comprises at least one intermediate wall or door, comprising a concrete material having a thickness of at least 1 meter and density of at least 2.35 g/cm³.

18. The method of claim 17, wherein the vault comprises at least one additional shielding layer selected from the group consisting of:
so an iron shielding of approximately 20 cm thickness on an interior side of the intermediate wall;
a borated polyethylene shielding of approximately 40 cm thickness on an exterior side of the intermediate wall; and
a borated polyethylene shielding of approximately 40 cm thickness on an interior side of an entrance door of the vault.

19. The method of claim 12, wherein a distance between an imaging isocenter of the patient during a treatment planning stage, and an imaging isocenter of the patient during a treatment application stage, is less than 2 meters.

20. The method of claim 12, wherein a distance between a distal end of the proton beam delivery device and a skin portion of the patient, is less than 1 meter.
